(19) United States
(12) Patent Application Publication
(10) Pub. No.: US 2024/0401120 A1
(43) Pub. Date: Dec. 5, 2024

(54) METHODS FOR BIAS-FREE QUANTIFICATION OF RNA

(71) Applicant: Chronix Biomedical, Irvine, CA (US)

(72) Inventors: Ekkehard Schütz, Nashville, TN (US); Julia Beck, Göttingen (DE)

(21) Appl. No.: 18/696,933

(22) PCT Filed: Sep. 28, 2022
(86) PCT No.: PCT/US2022/045074

§ 371 Date: Mar. 28, 2024

Related U.S. Application Data
(60) Provisional application No. 63/250,106, filed on Sep. 29, 2021.

(51) Int. Cl. C12Q 1/6844; C12N 9/12; G16H 50/20

(52) U.S. Cl. CPC C12Q 1/6844; C12N 9/1276; G16H 50/20; C12Y 207/07049

(57) ABSTRACT

This invention provides a method of precisely quantifying the amount of one or more RNAs of interest in a biological sample obtained from a subject.

### CROSS-REFERENCE TO RELATED APPLICATIONS

This application claims priority benefit of U.S. Provisional Application No. 63/250,106, filed Sep. 29, 2021, which is incorporated by reference for all purposes.

### BACKGROUND

PCT/US21/24231, which is incorporated by reference, describes methods for assessing cell free (cfDNA) to accurately determine target DNA concentrations (e.g., measured as copies/ml) and/or percentages of target DNA. However, fragmentation of RNA in biological samples due to degradation also presents problems in accurately quantifying levels of RNA in the samples, e.g., by RT-PCR expression profiling, to evaluate a physiological or pathophysiological condition of interest.

### BRIEF SUMMARY OF ASPECTS OF THE DISCLOSURE

The present disclosure provides methods and kits for assessing fragmentation of RNA, e.g., RNA from a tissue or bodily fluid sample. The methods thus allow precise determination of concentration of one or more RNAs of interest in a sample. Certain aspects of the disclosure are summarized below.

In one aspect, the disclosure provides a method to quantify a precise amount of an RNA of interest in a sample from a subject using two or more PCR reactions, the method comprising (a) providing a cDNA preparation obtained performing a reverse transcription reaction on RNA obtained from the sample; (b) performing a first PCR on the cDNA preparation to obtain a first amplicon, wherein the first PCR amplifies a first region of a cDNA for an RNA of interest to be quantified; (c) performing a second PCR on cDNA of (a) that amplifies a second region of the cDNA for the RNA of interest to obtain a second amplicon, wherein the second amplicon differs in length from the first amplicon by at least 10 base pairs; (d) quantifying the yield of the first amplicon; (e) quantifying the yield of the second amplicon; and (f) determining a precise amount of the RNA of interest present in the sample by interpolating the amplicon length of (b) and (c) to zero. In some embodiments, the first region amplified in (b) overlaps with the second region amplified in (c). In some embodiments, the first region amplified in (b) does not overlap with the second region amplified in (c). In some embodiments, the method further comprises determining the average length of the RNA in the sample. In some embodiments, the precise amount of a first target RNA in (d) is determined as a concentration. In some embodiments, a precise ratio between at least two expressed RNAs is determined. In some embodiments, the sample is an FFPE sample. In other embodiments, the sample is a cell-free blood or urine sample. Alternatively, in some embodiments, the sample is from a biopsy sample or surgically resected tissue. In some embodiments, the patient is a human. In some embodiments, the two or more PCR reactions are performed in a multiplex assay that amplifies at least two RNAs of interest to be quantified. In some embodiments, the method further comprises introducing the precise amounts of at least two RNAs of interest as input values in a multi-parametric computer model to generate a score from two or more RNAs for different genes. In some embodiments, the method further comprises predicting likelihood of the presence of a disease or disease condition from the score. In some embodiments, the method further comprises predicting likelihood of response to a therapy of a disease or disease condition from the score.

In a further aspect, the disclosure provides a kit comprising primers to amplify at least two amplicons of different lengths from cDNA reverse transcribed from an RNA of interest. In some embodiments, the kit further comprises reverse transcriptase, dNTPs, reaction buffers, and/or a polymerase to generate the at least two amplicons.

### BRIEF DESCRIPTION OF THE DRAWINGS

FIG. 1 shows a frequency graph of fragments sizes from different FFPE block samples. (Top line, Block2019Slice1; Second line from top, Block2019slice2; Third line from top; Block2013Slice1; Fourth line from top, Block2013Slice2; Bottom line: Block2013Slice3. The designations of lines from top to bottom for purposes of this brief description are based on the 200 bp distance point on the x-axis).

FIG. 2 shows a frequency graph of read counts over all reads assigned to any known mRNA sequences from different FFPE block samples. The arrow points to the trendline.

FIG. 3 provides a graph of a simulation of error rate due to RNA fragmentation in a multi-parametric expression profiling using targeted RT-PCR and dependence on primer selection. Upper line, Corrected; Middle line, 65-75 bps; Lower line, 85-99 bp

FIG. 4 provides a graph a simulation of error rate due to RNA fragmentation in a multi-parametric expression profiling using targeted RT-PCR and dependence of expression profiles. Upper line, Corrected; Middle line, 65-75 bps; Lower line, 85-99 bp

### DETAILED DESCRIPTION

As noted above, degradation of RNA presents problems in accurately quantifying RNAs of interest in a biological sample. The limited stability of RNA can be ameliorated, in part, using preservative solutions or other means of fixing a sample. In instances where tissue is employed, the material, e.g., obtained biopsies, is often fixed in formalin and then embedded in paraffin to generate a paraffin block suitable for microscopic inspection. Such formalin-fixed paraffin embedded (FFPE) samples can also be evaluated for RNA expression. The quality of RNA extracted from FFPE can vary widely, however, based on parameters such as time interval before fixing the sample, the size of the tissue (e.g., in larger samples it requires a longer period of time for the fixative agent, e.g., formalin, to diffuse into interior regions of the tissue sample), and variations in the fixative solution, e.g., formalin solution, such as pH, which influence RNA stability. Similarly, other biological samples for evaluations, e.g., including, but not limited to serum, plasma, or urine samples for evaluation of cell-free RNA (cfRNA), biopsy samples, surgical resection samples, or lavage samples, may have varying degrees of RNA fragmentation.

Generally, in any of these circumstances, the RNA is fragmented to sizes of unknown length and distribution. This phenomenon is thought to be counteracted for RNA profiling by using one or more reference genes, which works well if an untargeted whole transcriptome analysis is used, so that all RNA is counted independently of the varying length of a fragment. This changes significantly for PCR-based quantification, where PCR efficiency is reduced and the amount of this reduction is dependent on the degree of fragmentation of the RNA sample as well as on the respective PCR-amplicon length. Further, in this context in the majority of applications, the diagnostic result is obtained in a multi-gene approach, often as a result of a multi-parametric model to analyze expression levels of a panel of genes. In the simplest embodiment, it can be a linear regression with several different gene expression determinations as an independent variable and a certain outcome as a dependent variable. Such an outcome can be defined as a prognostic, diagnostic or predictive result and can be used as a continuous variable or stratified by several cut-off values or even dichotomized by a single cut-off value.

The disclosure thus provides methods and kits for precise quantification of target-specific RNA in tissue or other samples, e.g., bodily fluid samples.

### Terminology

In the context of the present disclosure samples to be evaluated typically contain fragmented RNA. As used herein, the terms “proportion of amplifiable RNA” or “fraction of amplifiable RNA” in a sample refers to the amount of an RNA of interest in a sample that can provide an amplified product of a size of interest.

The term “cell-free RNA” or “cfRNA” as used herein means free RNA molecules of 20 nucleotides or longer that are not contained within any intact cells. Often, “cfRNA” is evaluated in blood, e.g., can be obtained from human serum or plasma, but may be evaluated in any cell-free bodily fluid.

The terms “amplifying” and “amplification” generally refer to generating one or more copies (or “amplified product” or “amplification product”) of a nucleic acid. In typical embodiments, an amplified product is generated by polymerase chain reaction (PCR), which provides exponential amplification of a nucleic acid of interest using primer pairs and one or more nucleic acid polymerases. Thus, the term “polymerase chain reaction” as used herein refers to any method of exponential amplification performed with 5′ and 3′ primers that target a nucleic acid of interest and one or more nucleic acid polymerases. The term include multiplex reactions.

The term “primer” refers to an oligonucleotide that acts as a point of initiation of nucleic acid synthesis under conditions in which synthesis of a primer extension product complementary to a nucleic acid strand is induced, i.e., in the presence of four different nucleoside triphosphates and an agent for polymerization (e.g., DNA polymerase or reverse transcriptase) in an appropriate buffer and at a suitable temperature. A primer includes a “hybridizing region” exactly or substantially complementary to the target sequence, preferably about 15 to about 35 nucleotides in length. A primer oligonucleotide can either be composed entirely of the hybridizing region or can contain additional features which allow for the detection, immobilization, or manipulation of the amplified product, but which do not alter the ability of the primer to serve as a starting reagent for template-directed extension. For example, a nucleic acid sequence tail can be included at the 5′ end of the primer that hybridizes to a capture oligonucleotide.

The term “target sequence” or “target region” refers to a portion of an RNA of interest to be amplified for quantification. The term includes cDNA corresponding to the RNA of interest. A “target” RNA refers to an RNA of interest to be quantified.

The terms “precise amount” or “precisely quantified” refers to quantification of one RNAs of interest that corrects for fragmentation of the sample.

As used herein, the terms “nucleic acid,” “polynucleotide” and “oligonucleotide” refer to primers, probes, and oligomer fragments. The terms are not limited by length and are generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA. Oligonucleotides for use in the invention may be used as primers for amplification of a target of interest.

A nucleic acid, polynucleotide or oligonucleotide can comprise phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, polynucleotide or oligonucleotide can comprise the five biologically occurring bases (adenine, guanine, thymine, cytosine and uracil) and/or bases other than the five biologically occurring bases. These bases may serve a number of purposes, e.g., to stabilize or destabilize hybridization; to promote or inhibit probe degradation; or as attachment points for detectable moieties or quencher moieties. For example, a polynucleotide of the invention can contain one or more modified, non-standard, or derivatized base moieties, including, but not limited to, N6-methyl-adenine, N6-tert-butyl-benzyl-adenine, imidazole, substituted imidazoles, 5-fluorouracil, 5 bromouracil, 5-chlorouracil, 5-iodouracil, hypoxanthine, xanthine, 4-acetylcytosine, 5 (carboxyhydroxymethyl) uracil, 5 carboxymethylaminomethyl-2-thiouridine, 5 carboxymethylaminomethyluracil, dihydrouracil, beta-D-galactosylqueosine, inosine, N6 isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2 thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl) uracil, (acp3)w, 2,6-diaminopurine, and 5-propynyl pyrimidine. Other examples of modified, non-standard, or derivatized base moieties may be found in U.S. Pat. Nos. 6,001,611; 5,955,589; 5,844,106; 5,789,562; 5,750,343; 5,728,525; and 5,679,785, each of which is incorporated herein by reference in its entirety. Furthermore, a nucleic acid, polynucleotide or oligonucleotide can comprise one or more modified sugar moieties including, but not limited to, arabinose, 2-fluoroarabinose, xylulose, and a hexose.

A “subject” or a “patient” in the context of this invention is any individual that is to be evaluated for expression of RNAs of interest. In typical embodiments, the patient is a human. In other embodiments, the patient is a mammal, e.g., a murine, bovine, equine, canine, feline, porcine, ovine, caprine, or a primate.

As used herein, the singular forms “a,” “an” and “the” include plural referents unless the context clearly dictates otherwise. Thus, for example, reference to “a molecule” includes a plurality of such molecules, and the like.

The term “about” as used herein refers to the usual error range for the respective value readily known to the skilled person in this technical field, for example ±10%, or ±5%, are within the intended meaning of the recited value.

### Quantitative Assessment of RNA

The present disclosure, provides, at least in one aspect, methods of more accurately quantifying RNA of an interrogated target, for example, RNA from an FFPE sample. The methods employ at least two PCR reactions that generate amplicons of different lengths to assess the degree of fragmentation of target RNAs. Because multiple PCRs are employed, the methods as described herein provide improved quantification of RNA, e.g., determined as a precise concentration.

### Analysis of RNA in Biological Samples

In one illustrative embodiment, RNA is analyzed in a tissue sample that has been preserved in a fixative, such as formalin. As described herein, the degree of fragmentation can differ among RNA targets and among RNA molecules for the same target. In the present invention, at least two PCR reactions are performed with primers that generate amplicons of different lengths. The yields are then compared in the differing PCR reactions to provide an improved quantification of the concentration of an RNA of interest in the application.

Primers are typically selected that amplify a target region that comprises a sequence that is not found at the end of the genes, e.g., is not contained in the last 10% at the 5′ or 3′ end of the full-length sequence of the RNA of interest. In some embodiments, the target region is in the middle region of the gene, e.g., selected to target a region the is 70% of the full-length of the RNA and does not include the 5′ or 3′ end sequences. In some embodiments, the primers employed in the PCR reactions to generate amplicons of different length are selected to amplify regions comprising the same target sequence, but to generate amplicons of different lengths. In some embodiments, one of the primers in each primer set shares at least partial sequence identity such that the sequences in the target region to which the primers hybridize overlap. Thus, for example, a forward primer of a primer set to generate a shorter amplicon may hybridize to a nucleic acid sequence that at least partially overlaps with the nucleic acid sequence to which the forward primer that generates the longer amplicon hybridizes. In some embodiments, the primer sets for each amplicon share a common primer that hybridize to the same target sequence. For example, a forward primer of a primer set to generate a shorter amplicon may be the same primer sequence as the forward primer to generate a longer amplicon.

In some embodiments, the primers in each primer set are both different and hybridize to different sequences in an RNA of interest to be quantified. In some embodiments, the amplicons that differ in length are generated from two non-overlapping target regions. For example, one primer set may be selected to amplify a first target region of the RNA of interest, whereas a second primer set to generate a second amplicon that differs in size from the first amplicon may be selected to amplify a second target region of the RNA of interest that does not overlap with the first.

As noted above, primers are selected that provided amplicons of different lengths. In some embodiments, the amplicons differ by 10 base pairs in length. In typical embodiments, the amplicons different by at least 15 base pairs in length. In some embodiments, the amplicons differ by at least 20 base pairs in length, or may different aby at least 25, at least 30, at least 35, at least 40, at least 45, or at least 50 base pairs in length. In typical embodiments, the primers are selected that generate amplicons that differ by no more than 100 or 150 base pairs in length, or no more than 200 base pairs in length. In some embodiments, the difference in amplicon sizes is in the range from 10 to 200 base pairs in length. In typical embodiments, the amplicons differ in size from about 20 to about 150 base pairs in length.

In some embodiments, a control PCR reaction may be performed that further comprise “spike-in” RNA i.e., control RNA added to the starting sample obtained from a patient, e.g., a blood, plasma, or serum sample, to control for the efficiency of extraction of the RNA from the patient sample.

### Amplification of RNA

Reactions are performed on RNA obtained from a sample, which is typically reverse transcribed to provide cDNA for subsequent analyses. In some embodiments, RNA is quantified in an FFPE sample. In some embodiments, cfRNA is quantified in a body fluid, e.g., serum or plasma, from a subject. In some embodiments, the sample is from a patient that has cancer.

In some embodiments, digital PCR is performed in which a limiting dilution of the sample is made across a large number of separate PCR reactions so that most of the reactions have no template molecules and give a negative amplification result. Those reactions that are positive at the reaction endpoint are counted as individual template molecules present in the original sample in a 1 to 1 relationship. (See, e.g., Kalina et al. NAR 25:1999-2004 (1997) and Vogelstein and Kinzler, PNAS 96:9236-9241 (1999); U.S. Pat. Nos. 6,440,706, 6,753,147, and 7,824,889; each incorporated by reference.) In some embodiments, a digital PCR may be a microfluidics-based digital PCR. In some embodiments, a droplet digital PCR may be employed.

One of skill understand that amplification reactions other than RT-PCR may be employed. For example, in some embodiments, isothermal amplification reactions may be used.

### Quantification

The amplicons obtained for each of the PCR reactions can be evaluated using any known technology, including, for example, digital droplet PCR, high throughput sequencing technology, or a hybridization assay that employs capture probes.

In some embodiments, cDNA sequencing and analysis are used to analyze amplicons obtained from the PCR reactions. For example, DNA sequencing may be accomplished using high-throughput DNA sequencing techniques. Examples of next generation and high-throughput sequencing include, for example, massively parallel signature sequencing, polony sequencing, 454 pyrosequencing, Illumina (Solexa) sequencing with HiSeq, MiSeq, and other platforms, SOLID sequencing, ion semiconductor sequencing (Ion Torrent), DNA nanoball sequencing, heliscope single molecule sequencing, single molecule real time (SMRT) sequencing, MassARRAY R, and Digital Analysis of Selected Regions (DANSR™).

Any technology that employs targeted hybridization (e.g., primer oligonucleotides or hybrid capture oligonucleotides) for selection of any genomic position can be used to evaluate the amounts of each amplicon generated.

The methods provided herein correct for the bias in previous RNA quantification methods that do not fully account for varying fragmentation in RNA.

In some embodiments, the concentration is compared for the PCR reactions that generate amplicons of different lengths. In some embodiments, a concentration of an RNA of interest is determined using PCR reactions as described herein to improve quantification.

In one embodiment, the concentration of an RNA of interest is measured by performing at least two different PCRs that generate amplicons of different lengths. A linear correlation with amplicon length as the independent variable and the measured concentration of the RNA target as dependent variable is then calculated. A precise concentration of the target RNA (which takes into account the degree of fragmentation as described herein) can be calculated by interpolation of the regression to an amplicon length of zero bp.

In another embodiment, precise concentrations of two or more RNA targets of interest are independently determined by generating two different amplicons of different lengths specific for each possible target of interest. In this illustrative embodiment, RT-PCRs from any analyzed gene-specific RNA with at least two different amplicon lengths can be used to calculate a precise concentration of mRNA. As above, the amplicon lengths of at least two different sizes would be used as independent variables and the concentrations of each of such RT-PCR as dependent values in a linear regression analysis. The interpolation to the intercept would then result directly in a precise concentration of the RNA of interest.

In another embodiment, the amplifiable fraction of the total RNA is determined and used to correct the measured concentrations of all possible targets of interest. In a first step, the average length of the total RNA needs to be determined. In a second step the amplifiable fraction of the total RNA (θRNA) is calculated for each amplicon length used in the sample. The interpolation of the values into zero bp (e.g., the intercept of a regression line) gives a precise value of the target. This can be deduced from the equation

$$RNA observed = RNA present \times \frac{RNA length - Amplicon length}{RNA length} = RNA present \times ( 1 - \frac{Amplicon length}{RNA length} )$$

(see, e.g., U.S. Patent Application Publication No. 20170327869) since the term solves to 1 only with 0 bp.

Thus, the following equation can be used to calculate the mean RNA length from the aforementioned linear regression:

$$mean RNA length = - \frac{precise RNA amount}{slope RNA}$$

Therefore, in one embodiment, the following formula can be applied to each result of one or more target PCRs with different amplicon lengths on an individual sample and for individual RNAs (RNA (i)) with an individual Amplicon length (Amplicon (i) length):

$$precise RNA ( i ) amount = RNA ( i ) measured \times \frac{mean RNA length}{mean RNA length - Amplicon ( i ) length}$$

### Multi-Parametric Analysis

The precise amounts of RNA determined as described herein can be used for any diagnostic, prognostice, or predictive application. For example, one embodiment, a linear regression can be performed using several different gene expression determinations as an independent variable and a certain outcome as a dependent variable. In some embodiments multi-parametric analysis can be performed in which RNA expressed by multiple genes is expressed. Such multi-parametric analyses are well known in the art (see, e.g., by way of illustration, Blok et al, Cancer Treatment Reviews 62:74-990, 2018, which provides a review of commercial gene expression profile analyses for invasive early breast cancer as an example). The precise concentrations of RNA determined as described herein can be employed in multi-parametric analyses using continuous variables or stratified by several cut-off values, or dichotomized by a single cut-off value.

### Computer Analysis

In some embodiments, the present invention provides systems related to the above methods of the invention. In one embodiment the invention provides a system for accurately assessing RNA levels in a sample comprising: (1) a sample analyzer for executing the method of accurately assess RNA levels in a sample comprising fragment RNA using at least two RT-PCR reactions that generate amplicons of different length to calculate the amplifiable fraction of a first amplicon generated by a first RT-PCR reaction in the sample and the amplifiable fraction of a second amplicon generated by a second RT-PCR reaction in the sample as described above; (2) a computer system for automatically receiving and analyzing data obtained in step (1) to calculate the fraction of amplifiable RNA from the first RT-PCR and the fraction of amplifiable RNA from the second RT-PCR in the sample.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 8, Windows™ 7, Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™ or Microsoft™ Explorer™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out the analysis and correlating functions as described above. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

### Kits

In a further aspect, the disclosure provides kits and composition for determining precise amounts of one or more RNAs of interest in the sample. In some embodiments, such a kit comprises primers to generate at least two amplicons from cDNA transcribed from an RNA of interest to be quantified that is present in a biological sample. In some embodiments, a kit further comprises a reverse transcriptase. In further embodiments, the kit can comprise reagents for performing PCR, including, but not limited to a polymerase, dNTPs, and buffers.

The following examples are provided by way of illustration only and not by way of limitation. Those of skill in the art will readily recognize a variety of non-critical parameters that could be changed or modified to yield essentially similar results.

### EXAMPLES

The following examples describes the development of an improved quantitative assay that provides precise quantification of RNA

### Example 1. Size Distribution of RNA from Different FFPE Blocks

### Materials and Methods

RNA was extracted from FFPE embedded tumor tissue using the AllPrep DNA/RNA FFPE Kit (QIAGEN). Two different specimens with different storage periods were used, one specimen was embedded in 2013 and the second in 2019, resulting in storage times at room temperature of 7 years versus 1 year. For each block, several slices were analyzed, where the first ones had a higher exposure to oxidation during storage periods compared to the slices that were collected from the deeper compartments of the FFPE block.

The total extracted RNA was subjected to rRNA depletion (NEBNext rRNA Depletion Kit (Human, Mouse, Rat), New England Biolabs) and sequencing libraries were prepared using the NEBNext Ultra II RNA Library Prep Kit for Illumina (New England Biolabs). Sequencing was conducted using a NextSeq550 (Illumina). After demultiplexing, the sequences were aligned to the human reference genome (HG19) after removal of duplicate reads the sequences were analyzed using the RSeQC program package (Wang et al., Bioinformatics 28:2184-2185, 2012)

### Results

### Inner Distance (or Insert Size) Between Two Paired RNA Reads

The inner distance is the mRNA length between two paired fragments. We first determine the genomic (DNA) size between two paired reads:

D_size=read2_start−read1_end, then
if two paired reads map to the same exon: inner distance=D_size;
if two paired reads map to different exons: inner distance=D_size−intron_size;
if two paired reads map to non-exonic regions (such as intron and intergenic region): inner distance=D_size;

The inner_distance may be a negative value if two fragments overlapped.

Thus, the resulting RNA fragment size is inner distance+read length (75 bp)

TABLE 1
Mean, standard deviation (SD) and median of the RNA fragment size detected in the different samples.

| | Block2013Slice1 | Block2013Slice2 | Block2013Slice3 | Block2019Slice1 | Block2019Slice2 |
|---|---|---|---|---|---|
| Mean (bp) | 129.6 | 121.6 | 113.5 | 160.3 | 151.7 |
| SD | 63.0 | 40.5 | 31.1 | 52.6 | 50.8 |
| Median | 112.5 | 112.5 | 112.5 | 152.5 | 142.5 |
| >100 bp (%) | 57.7 | 63.5 | 59.4 | 88.4 | 84.2 |

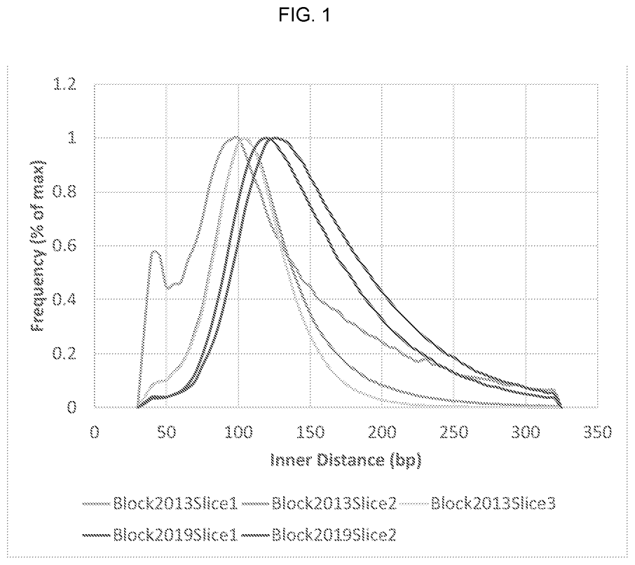

A frequency plot of the fragment sizes in the different samples is provided in FIG. 1.

This example demonstrates the uncertainty of fragmentation of FFPE derived RNA, where a substantial difference was observed comparing blocks; and a smaller, but still plainly evident, difference can be found within one FFPE block. The latter observation is thought to be due to aging of such blocks resulting in a deterioration and fragmentation of RNA from the block surface into the inner regions further to environmental influences such as oxygen exposure.

GC content was also calculated for all reads with a minimum mapping quality of 30 and, as expected, did not show any significant differences.

TABLE 2
GC-content for reads of different samples

| | Block2013Slice1 | Block2013Slice2 | Block2013Slice3 | Block2019Slice1 | Block2019Slice2 |
|---|---|---|---|---|---|
| Mean | 48.85 | 49.28 | 48.88 | 47.27 | 48.06 |
| SD | 14.60 | 13.20 | 12.81 | 13.77 | 13.76 |
| Median | 48.65 | 48.65 | 48.65 | 45.95 | 47.37 |

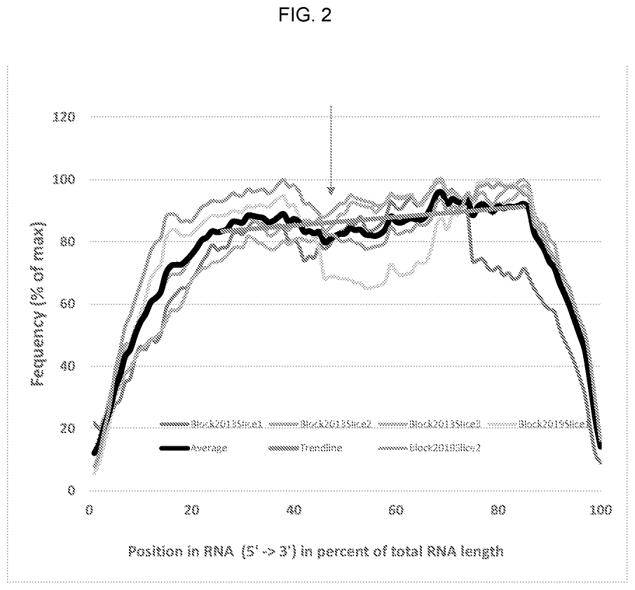

RNA read counts were also calculated over all reads assigned to any known mRNA sequences from different FFPE block samples. The results are shown in FIG. 2. A significant dependency on position was detected with p=0.03 (slope of regression). As a consequence, any PCR-based RNA quantification should be performed using a PCR design directed against the inner region of the transcript, preferentially more towards the 3′ end of the transcript, for example, at least about 75% or greater of the total transcript length, but not more than 90%.

Transcript integrity numbers (TINs) were calculated for 61177 annotated transcripts (HG19). The TIN metric reflects the degradation of each transcript and is calculated by measuring the evenness of coverage across the entire length of transcript by Shannon's entropy as described in Wang et al., BMC Bioinformatics 17:58, 2016. Transcripts with TINs of greater than zero in all five samples were selected and correlations were calculated for all sample pairs, the resulting Pearson r-values are provided in Table 3. The strength of association between different sample pairs varies significantly, even for RNA samples that were prepared from the same FFPE-block but from different slices.

TABLE 3
Pearson r-values for TIN correlations between different samples using transcripts
for which a TIN > 0 was obtained for all samples (n = 10702).

| | | | | |
|---|---|---|---|---|
| Block2013Slice1 | Block2013Slice2 | Block2013Slice3 | Block2019Slice1 | Block2019Slice2 |

| | | | | |
|---|---|---|---|---|
| Block2013Slice1 | 0.56 | 0.58 | 0.46 | 0.45 |
| Block2013Slice2 | | 0.89 | 0.84 | 0.84 |
| Block2013Slice3 | | | 0.73 | 0.73 |
| Block2019Slice1 | | | | 0.94 |

In summary, the results presented above demonstrated significant variability in RNA quality, i.e., fragment length, after extraction from FFPE-embedded tissue. As noted, such differences can even occur between different sampling areas (slices) from the same tissue block.

### Example 2 Effect of RNA Fragmentation on Expression Modelling Results

For this example, we assume the final result of a targeted expression profiling is based on a linear multi-parametric regression with 12 target and 2 reference genes. Each expression of a target gene (Eₙ) is first corrected by the measured expression (Eᵣ) of the reference genes (average) and the corrected value of each of the 12 gene expressions is used as independent variable in the model as y=a₁+x1+a₂*x2+ . . . aₙ*xₙ+b, with aₙ=factor for corrected expression value of geneₙ and Xₙ=corrected expression value of geneₙ; n=total number of target genes; b-model intercept (fixed value). We assume all PCRs are directed towards a region in the RNA, which is not highly variable, such as around the middle of the transcript. For the following simulations, 6 of the 12 expression factors (aₙ) are assumed to be of a positive value and 6 were set to a negative value, but were kept constant for all simulations.

In silico simulations were calculated to exemplify the error that will occur based on fragmentation of RNA, which per se is unknown. The simple approach to use the expression value of a reference gene to judge the quality is not sufficient, since it is grossly influenced by the amount of total RNA, efficiency of the reverse transcriptase step and other confounding factors, such as incomplete de-paraffinization. Those are usually statistically controlled be following operation procedures rigorously but can and will fail on an individual sample. Effects of different PCR efficiencies due to different RT-PCR amplicon lengths between target and reference gene are always contributing to a bias, which is exemplified in the following examples.

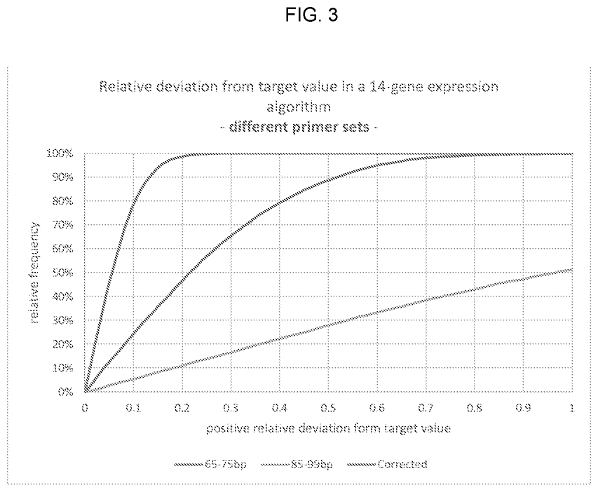

For each of the simulations, 30,000 individual data points were simulated under different assumptions. The first example (FIG. 3) shows the results of a simulation, where the precise expression (transcript count) of each of the 14 genes (Eₙ and Eᵣ) was set to a fixed value. We assume that RT-PCRs are used, which amplify the target using a narrow span of RT-PCR amplicon length. The length of the RT-PCR were randomized in two ranges with a narrow span of 10 and 14 bp and used to calculate the final model result whereas the deviation was calculated against unfragmented RNA (negative deviation were converted to absolute values for display). The fragmentation of RNA was assumed to be between 100 and 300 bp, which—as said—is usually the unknown underlying confounding factor of such multi-parametric assays.

The two RT-PCRs that are simulated for each gene in the two size ranges were used to correct the model result by using the approach described for DNA. Briefly, for each data point and gene, the amplicon lengths and the gene simulated expression were subjected to a linear regression with the amplicon lengths as independent and the expression values as dependent variables. The interpolation of the two results to the intercept was used as Y=f(X)ₙ, with X=0 for the “corrected” values

If we assume that a dichotomized two-state model (e.g. high risk vs. low risk) is the final, e.g., prognostic, interpretation, 50% of the results will show a deviation of 20% for the short range PCRs and 90% for the longer amplicon RT-PCRs. This decreases to about 1% if the correction is used. This simulation also includes the error propagation due to two RT-PCRs with their technically unavoidable imprecision.

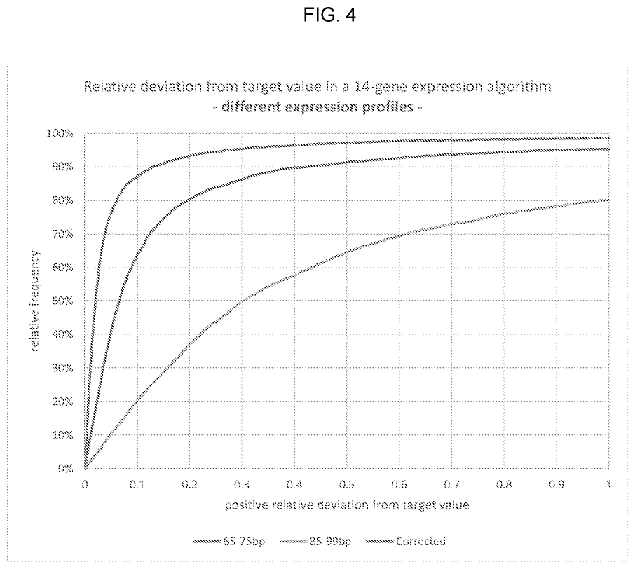

The second example (FIG. 4) is calculated for a fixed primer set, but using different possibly occurring expression profiles, which are simulating different samples. The calculation are—other than that—performed as described above.

As shown in FIG. 4, the 20% error will occur in 20% of sample when the short amplicon RT-PCRs are used and 63% of the samples if the longer amplicon RT-PCRs were used. The correction again results in a highly significant reduction of erroneous results to about 5%.

All accession numbers, patents, patent applications, and other published reference materials cited in this specification are hereby incorporated herein by reference in their entirety for their disclosures of the subject matter in whose connection they are cited herein.

1. A method to quantify a precise amount of an RNA of interest in a sample from a subject using two or more PCR reactions, the method comprising
(a) providing a cDNA preparation obtained performing a reverse transcription reaction on RNA obtained from the sample;
(b) performing a first PCR on the cDNA preparation to obtain a first amplicon, wherein the first PCR amplifies a first region of a cDNA for an RNA of interest to be quantified;
(c) performing a second PCR on cDNA of (a) that amplifies a second region of the cDNA for the RNA of interest to obtain a second amplicon, wherein the second amplicon differs in length from the first amplicon by at least 10 base pairs;
(d) quantifying the yield of the first amplicon;
(e) quantifying the yield of the second amplicon; and
(f) determining a precise amount of the RNA of interest present in the sample by interpolating the amplicon length of (b) and (c) to zero.

2. The method of claim 1, wherein the first region amplified in (b) overlaps with the second region amplified in (c).

3. The method of claim 1, wherein the first region amplified in (b) does not overlap with the second region amplified in (c).

4. The method of claim 1, further comprising determining the average length of the RNA in the sample.

5. The method of claim 1, wherein the precise amount of a first target RNA in (d) is determined as a concentration.

6. The method of claim 1, wherein a precise ratio between at least two expressed RNAs is determined.

7. The method of claim 1, wherein the sample is an FFPE sample.

8. The method of claim 1, wherein the sample is a cell-free blood or urine sample.

9. The method of claim 1, wherein the sample is from a biopsy sample or surgically resected tissue.

10. The method of claim 1, wherein the patient is a human.

11. The method of claim 1, wherein the two or more PCR reactions are performed in a multiplex assay that amplifies at least two RNAs of interest to be quantified.

12. The method of claim 1, further comprising introducing the precise amounts of at least two RNAs of interest as input values in a multi-parametric computer model to generate a score from two or more RNAs for different genes.

13. The method of claim 12, further comprising predicting likelihood of the presence of a disease or disease condition from the score.

14. The method of claim 12, further comprising predicting likelihood of response to a therapy of a disease or disease condition from the score.

15. A kit comprising primers to amplify at least two amplicons of different lengths from cDNA reverse transcribed from an RNA of interest.

16. The kit of claim 15, further comprising reverse transcriptase, dNTPs, reactions buffers, and/or a DNA polymerase to generate the at least two amplicons.
